(19) United States
(12) Patent Application Publication
(10) Pub. No.: US 2026/0108329 A1
(43) Pub. Date: Apr. 23, 2026

(54) METHOD AND ANALYSIS SYSTEM FOR CREATING A DIGITAL 3D MODEL OF THE DENTITION OF A PATIENT

(71) Applicant: BITE-FINDER AG, Liestal (CH)

(72) Inventors: Björn ZÄUNER, Liestal (CH); Mike ZÄUNER, Liestal (CH); Markus HOFMANN, Liestal (CH)

(21) Appl. No.: 19/153,978

(22) PCT Filed: Feb. 6, 2024
(86) PCT No.: PCT/EP2024/052935

§ 371 Date: Aug. 6, 2025

(30) Foreign Application Priority Data
Feb. 7, 2023 (DE) 10 2023 102 973.8

(51) Int. Cl. A61C 9/00; G16H 50/50

(52) U.S. Cl. CPC A61C 9/004; G16H 50/50

(57) ABSTRACT

A method for creating a digital 3D model of a patient's dentition, according to the invention, provides that in a computing unit for a plurality of dentition variants, each comprising a combination of a digital 3D model of the upper jaw and a digital 3D model of the lower jaw of the patient, which differ from each other in the relative positioning of the upper and lower jaws, respectively:
the digital 3D model of the upper jaw is combined with the digital 3D model of the patient's lower jaw in such a way that the upper and lower jaws rest on each other at a number of contact points without any spatial overlap of parts of the upper and lower jaws, and
the contact surface between the upper and lower jaw is determined, whereby an optimized denture variant is selected as a 3D model of the denture, taking into account the contact surface.

The invention relates to a method for creating a digital 3D model of a patient's dentition. It further relates to an analysis system suitable for carrying out the method, in particular for use in the planning of dental prostheses.

When planning dental prostheses, for orthodontic planning or in other areas of modern dental technology, in particular to improve the basis for decision-making or the starting point for the use of CAD/CAM processes in dentistry and dental technology, the conditioning and analysis of a patient's initial dental situation or an intermediate or final check of a therapeutic treatment may be based upon existing or digitally recorded data that accurately reflect the dental situation.

In modern dental care concepts, for example, prostheses such as crowns and/or implant-supported dentures, bridges, or similar devices are usually fabricated by reproducing the patient's oral situation as accurately as possible in order to achieve the highest possible fit and, in addition to the desired medical effects, the highest possible wearing comfort for the patient. In conventional treatments, it has been common practice to take an impression of the patient's teeth using a type of kit, impression material, or other hardening substance to create a negative mold of the actual situation. This can then be used to make a plaster model, for example. This plaster model can then be used as a basis for planning the fabrication and insertion of dental prostheses. These can then be digitized, for example using an extraoral 3D scanner, so that the dental work can subsequently be fabricated using digital technologies (CAD/CAM).

In more modern concepts, the patient's oral situation is recorded digitally, for example using intraoral scanners to capture three-dimensional patient data that reflect the patient's oral situation. This three-dimensional data can then be used to create 3D models of the patient's teeth (e.g., as 3D prints), which can then be used to plan the treatment strategy and dentures for the patient using digital methods, which is significantly faster and more cost-effective than before. In particular, as a result of such digital planning, the required dental prosthesis can be manufactured automatically using transferable 3D data.

However, these digital methods are currently subject to a serious limitation. Due to the nature of the technology and the technical capabilities and limitations of the scanners used to capture 3D patient data in the patient's mouth, it is only possible to capture the patient's upper jaw and lower jaw separately from each other. After scanning the oral situation, for example using an intraoral scanner, the data is therefore only available in the form of a digital 3D model of the patient's upper jaw on the one hand and a digital 3D model of the patient's lower jaw on the other. For the proper reproduction of the patient's entire oral situation, these two partial models must be combined in such a way that they correspond as closely as possible to the actual oral situation.

This combination of the digital 3D model of the upper jaw and the digital 3D model of the patient's lower jaw is referred to as the “bite.” The bite is the essential starting point for continuing all further steps in dental technology at a reasonable level and with a level of quality that is acceptable to the patient. The technical devices used for this purpose to date, such as intraoral and extraoral 3D scanners for capturing digital tooth models, do not generate sufficiently accurate bites.

The current technical starting point is the digitization of individual jaws, followed by the manually aligned bite. The individual jaw scans are then compared (matched) with the bite scan in order to align them accordingly. A Z-axis shift is usually used to eliminate any intersections or overlaps between the two jaws. However, this approach is unsatisfactory in principle because the jaw can be shifted not only along the Z-axis but also along the condylar path, making the approach incomplete. This leads to a high error rate in the subsequent process chain from CAD (design) to CAM (production).

The invention is therefore based on the task of to provide a method for creating a digital 3D model of a patient's dentition, in particular for use in planning dental prostheses, with which a complete digital 3D model of a patient's dentition can be created in a particularly reliable and high-quality manner using existing digital 3D models of both the upper and lower jaw of the patient. Furthermore, an automatic analysis system particularly suitable for carrying out the method is to be specified.

With regard to the method, this task is solved according to the invention by combining, in a computing unit, for a plurality of dental variants, each comprising a combination of a digital 3D model of the upper jaw with a digital 3D model of the lower jaw of the patient, which differ from one another in the relative positioning of the upper and lower jaws, in each case:
the digital 3D model of the upper jaw is combined with the digital 3D model of the patient's lower jaw in such a way that the upper and lower jaws rest on each other at a number of contact points without any spatial overlap of parts of the upper and lower jaws (often referred to in practice as “penetration”), and
the contact surface between the upper and lower jaw is determined, wherein an optimized denture variant is selected as a 3D model of the denture, taking into account the contact surface.

The invention is based on the consideration that the dental model composed of the two components, upper jaw and lower jaw, can be assumed to have a particularly high “accuracy of fit” and thus to reproduce the actual oral situation of the patient as realistically as possible if the two components, upper jaw and lower jaw, fit together as closely as possible. The contact surface over which these components come into contact with each other is therefore considered, according to one aspect of the invention, to be a particularly suitable criterion for accuracy of fit and is taken into account accordingly.

According to an aspect of the invention that is considered to be independently inventive, the static friction that results from a planned displacement of the upper and lower jaw components relative to each other can also be used as a basis for determining the accuracy of fit. This is essentially described by the contact surface, although for a more refined evaluation, depending on the angle of inclination of a respective surface element relative to the intended direction of displacement of the components relative to each other, an individualized weighting of the contributions of the respective surface element to the static friction can be carried out. Surface elements with a comparatively strong inclination relative to the direction of displacement can, for example, be weighted with an increased contribution to static friction, since their shape alone means that they offer more resistance to the intended displacement than comparatively flat surface elements.

According to an aspect of the invention that is considered to be independently inventive, a plurality of dentition variants corresponding to a temporal sequence within a chewing movement can be taken into account for the selection of the 3D model of the dentition. This is intended in particular to ensure that not only static aspects, but also dynamic aspects, i.e., those occurring during the chewing movement, are taken into account in the selection of the 3D model. Surprisingly, it has been found that these aspects can be particularly important for the care of the patient and their perceived comfort when wearing a custom-made prosthesis.

According to a further aspect of the invention, different contributions to friction between the teeth can be suitably taken into account. The contributions to friction can, for example, include contact friction (tooth enamel), fluid friction (saliva), and form-related interlocking effects.

Advantageously, the evaluation of the dentition variants also takes into account those that differ in the inclination of the upper jaw relative to the lower jaw. This allows the six degrees of freedom to be taken into account when combining the two elements, the upper jaw and the lower jaw.

According to one aspect of the invention, the dental variant for which the contact surface between the upper and lower jaws assumes a maximum value can be selected as the 3D model of the dentition.

The creation of the dentition variants and/or the determination of the dentition variant to be selected is particularly preferred using artificial intelligence. This is based on the consideration that the determination of an optimal locking point or the optimal combination of the upper and lower jaw models with each other is difficult to achieve using conventional modeling due to the complicated friction relationship between the teeth (static friction, fluid friction, interlocking effects, etc.) and is therefore provided for in accordance with one aspect of the invention, a neural network is used to learn the required friction conditions on the basis of examples.

Using artificial intelligence, an occlusion algorithm can apply various modern techniques from computer graphics to the 3D models of the tooth structures in a preliminary analysis step, in accordance with aspects of the invention that are considered to be independently inventive. For this purpose, filters can be used to detect extrema and edges, and selective smoothing can be used with the aid of partial differential equations to detect relevant tooth areas. In addition, principal component analysis and pattern recognition can be used to identify relevant geometries. The main objective of the preliminary analysis is to extract as much information as possible about patterns, edges, and geometric structures from the 3D model.

Based on the information obtained in the preliminary analysis, a hypercube-based algorithm for the neuroevolution of augmentation topologies can be trained in a second step according to one aspect of the invention. For example, 1875 training examples are used to teach the algorithm how to calculate occlusion accurately. According to one aspect of the invention, the key concept in this second step is to train a neural network to determine the correct occlusion. Throughout the learning process, the neural network is advantageously expanded so that it can adapt its topology. This flexibility enables it to effectively use and combine the information obtained during the preliminary analysis, thereby increasing the neural network's freedom in learning and calculating the correct occlusion.

Various filters and techniques from the field of computer graphics can be used in accordance with aspects of the invention to extract relevant information from 3D models. Subsequently, a special neural network is advantageously trained to calculate the occlusion based on the extracted information.

In addition to AI-driven evaluation of static occlusion, dynamic occlusion can be determined through AI-supported analysis of tooth facet shapes. Here, too, an algorithm based on hypercubes is advantageously used for the neuroevolution of augmentation topologies. In addition, possible jaw joint movements can be described by differential algebraic inequality systems. From a Bayesian statistical point of view, the differential algebraic inequality systems form a priority distribution. According to one aspect of the invention, this distribution is continuously refined by the Al using the information derived from the tooth shape, thus gradually approaching the limits of what is possible in terms of information theory.

With regard to the automatic analysis system for use in the planning of dental prostheses, the aforementioned task is solved by comprising the following:
a first computing device comprising a memory that stores non-transitory instructions which, when executed by one or more processors of the first computing device, cause the first computing device to:
read out the digital 3D models of the upper jaw and the lower jaw from a second computing device comprising a mass storage device in which a digital 3D model of a patient's upper jaw and a digital 3D model of the patient's lower jaw are stored,
for a plurality of dental variants, each comprising a combination of the digital 3D model of the upper jaw with the digital 3D model of the patient's lower jaw, which differ from one another in the relative positioning of the upper and lower jaws, in each case:
   combine the digital 3D model of the upper jaw with the digital 3D model of the patient's lower jaw in such a way that the upper and lower jaws rest on each other at a number of contact points without any spatial overlap of parts of the upper and lower jaws, and
   determine the contact surface between the upper and lower jaw, and, taking into account the contact surface, select an optimized dentition variant as a 3D model of the dentition.

Preferably, when executed by one or more processors of the first computing device, the non-transitory commands cause the first computing device to consider dentition variants that differ from each other in the inclination of the upper jaw relative to the lower jaw.

Preferably, when executed by one or more processors of the first computing device, the non-transitory commands cause the first computing device, according to one aspect of the invention, to select the dentition variant as a 3D model of the dentition for which the contact surface of the upper and lower jaws assumes a maximum value.

In particular, according to one aspect of the invention, a concept is provided that fully automatically simulates occlusion determination and finds the most perfect bite possible between two digital tooth models. The underlying algorithm preferably processes two digital models (for the upper and lower jaw, in any orientation) and determines the perfect bite by simulating jaw movements. For alignment, according to one aspect of the invention, a self-learning algorithm is used which finds a basic alignment based on many digital models.

In one aspect of the invention, it may be provided to position the models in a 3D image (DVT, MRI, CT) specific to the patient and to calculate a joint position based on the result calculated by the preceding algorithm of static occlusion.

According to an aspect considered to be independently inventive, the 3D model of the dentition determined and selected according to the concept described above can be used as the basis for the fabrication of a dental prosthesis.

In advantageous embodiments, further algorithms can be used individually or in combination with each other as desired, which:
Repair models
Close holes, remove or reduce artifacts
Reduce file size with virtually no loss using a proprietary compression algorithm (faster CAD processing, storage efficiency, etc.)
Socket/scan to print
Models are sealed watertight and the file name is engraved (+aligned) so that they can be sent directly to a 3D printer for production of a spatial model
Dynamic occlusion
Perform a simulation of all possible mandibular movements based on the Bennett angle, condylar trajectory, and grinding facets. According to one aspect of the invention, the movement (“ISS,” Immediate Side Shift) can also be taken into account. The output can advantageously be in XML file format, enabling further processing in CAD software to design dentures and take individual movement into account
Bite elevation (VDO) can be performed after the calculation, whereby jaw movements can be taken into account at the same time. This can be an important component for the design of splints, tabletops, or other restorations/appliances that require/positively influence bite elevation.

The advantages achieved with the invention are, in particular:
Significant increase in efficiency in the laboratory and dental practice (=less time loss)
Significant reduction in the reject rate, leading to lower production costs and lower material consumption
Increased level of automation (=lower costs, faster delivery)
Countermeasure to skilled labor shortages, as the software is easy to use and offers automatic quality control
Significant increase in quality (no multiple visits or follow-up treatment for patients at the dentist because the dentures did not fit properly)
Missing piece of the puzzle for a complete digital workflow (currently still analog intermediate steps in the laboratory with manual adjustments)

According to further aspects, each of which is considered to be independently inventive, the concept described can be supplemented by:
Integration of 2D and/or 3D X-ray images of the patient's dental situation. This allows information intended for use in artificial intelligence in particular to be enriched and supplemented, so that the results can be further improved.
Integration of clinical photos or 3D facial scans to determine the joint position with sufficient accuracy
Use of static and dynamic data to check final CAD designs as an automatic quality control and to assign them an evaluation scale, which then initiates further automatic or manual steps.
Adaptation of the 3D base through dynamics and statics to automatically dock to a physical articulator.
Simulation of further chewing movements
Calculation of bite force using an algorithm
Automatic integration of additional patient-related data or files (new scans, scanbodyscan, etc.)
Creation of an occlusion report with comparison of contact points (preferably before/after)
Specifications/instructions for the subsequent design
Automatic optimization of the crown position based on the contacts and dynamics
Simulation of crown movement and span in the lower jaw through dynamics
“4D JawMovement over time.” Simulation of jaw movements over several years to visualize the effects on the tooth substance (this can show the patient the effects of grinding over time and motivate them to take further treatment steps, such as a grinding splint)
Creation of a desired intercuspidation/contact point distribution and, based on this, a grinding protocol (2D or as a 3D file for production) to eliminate the early contacts that prevent this situation from becoming the status quo.
Use of the habitual jaw movements from the tooth situation or prosthesis situation to develop follow-up prostheses that are customized
Automatic tabletop design based on the situation and jaw movements to define new bite positions.
Quality control of models (scanning of 3D models and automatic comparison and calculation of a score value with the 3D CAD model)
Monitoring (comparison and recommendations for action based on models over time, e.g., during annual dental visits. Indication of changes, grinding facets, etc.)

Further advantageous aspects of the invention may include, for example, in the form of corresponding modules or integrated functionalities:
A “save-by-transformation” module: This can offer the option of reading only the vertices from 3D formats and manipulating them with rotation matrices. In practice, the rotation matrices are precisely the calculated transformations required to bring the models into occlusion. The idea considered to be independently inventive here is that the 3D format does not have to be completely re-saved, as additional information such as color information may be lost in the process. It is also possible to transform “attached” models, such as bridges, etc., into occlusion.
The provision of marking functions in the GUI (graphical user interface): this allows the user to specify whether certain areas should be in contact or ignored. This gives the user the opportunity to influence the occlusion calculation; for example, they can mark the gums and thus allow greater penetration in the gum area, etc. (gums give way when bitten).
An option to adjust the penetration depth: This allows the user to set the maximum penetration depth and thus create more/larger or fewer/smaller contacts.
An option to perform bite elevation based on dynamic occlusion data.
Provision of a report (e.g., as a video) that compares the before and after situations based on the contact points and evaluates them using indicators.

In particular, different occlusion algorithms can be provided, from which the user can choose:
If the model is already close to occlusion, a local algorithm can be used to restrict the allowed movement of the lower jaw to the occlusion position.
If the model is far from occlusion, a global algorithm can be used that does not restrict the permitted movement.

The “Bite-Finder” concept described above thus enables the upper and lower jaws to be scanned using an intraoral scanner with the appropriate dentition without the third scan of the occlusion that would otherwise be necessary. “Bite-Finder” is therefore an innovative concept that simplifies the creation of digital 3D models of a patient's bite, thereby transforming dental practices and laboratories. This technology is revolutionizing the dental industry by providing a more efficient, accurate, and cost-effective method for bite analysis, adjustment, and treatment planning. According to aspects of the invention, it comprises the main components:

Bite Data Acquisition:
“Bite-Finder” loads digital 3D models of both the upper and lower jaw of a patient. This data can be captured using various means, such as intraoral scanners or 3D dental imaging technologies.

AI-supported alignment:
One aspect of Bite-Finder that is considered to be independently inventive is its AI-powered alignment features. Once the digital models of the upper and lower jaw are available, the system uses sophisticated algorithms to align these models with unparalleled precision. Unlike traditional methods, which rely solely on adjusting the Z-axis, Bite-Finder takes into account various degrees of freedom, including translation and rotation, ensuring that the upper and lower jaws fit together seamlessly.

Occlusion and penetration analysis:
According to one aspect of the invention, Bite-Finder performs a detailed analysis of the aligned models to identify occlusion and penetration problems. Through comprehensive simulations and calculations, the system determines the optimal bite position by adjusting the contact points between the teeth. This process ensures that the patient's bite is in perfect harmony, resulting in improved comfort and better treatment results.

Dynamic bite simulation:
In addition to static bite adjustments, one aspect of the invention offers the possibility of dynamic bite simulation. It can replicate patient-specific jaw movements so that dentists and dental technicians can assess how the bite works during real-life activities such as chewing and speaking. This feature increases the precision of restorative treatments and the design of appliances.

Automated model improvement:
Bite-Finder goes beyond bite corrections. It can repair models, close holes, remove artifacts, reduce file size using proprietary compression algorithms, and even prepare models for 3D printing. These features contribute to a streamlined and efficient workflow.

Quality control and assurance:
To maintain high quality standards, Bite-Finder has automatic quality controls. This ensures that bite adjustments and models meet industrial and clinical standards.

In addition, predictions about the development of the grinding facets or wear are possible.

The particular benefits and advantages of the invention can be seen in particular in:
Accuracy: AI-driven alignment and analysis results in highly accurate bite corrections and reduces the risk of rework and errors.
Efficiency: By automating complex processes, Bite-Finder significantly reduces the time required for bite analysis and treatment planning.
Cost efficiency: Fewer manual adjustments, less material waste, and a more efficient workflow result in cost savings for dental practices and laboratories. Patient comfort: Thanks to precise bite adjustment, patients experience greater comfort during treatment.
Versatility: Bite-Finder can be used in various dental specialties, from general dentistry to orthodontics and prosthetics.

The Bite-Finder concept, as described in one aspect of the invention, is a groundbreaking innovation that brings efficiency, precision, and affordability to the dental industry. By combining digital technology, Al algorithms, and 3D modeling, it simplifies the process of creating accurate 3D bite models, which ultimately improves the quality of patient care and dental treatments. With “Bite-Finder,” dentists and dental technicians can work more effectively and offer their patients excellent dental solutions.

Process:
View preview and send to lab for use (and payment)

The following can be considered inventive according to one aspect of the invention:
A method for creating a digital 3D model of a patient's bite, comprising:
a. Capturing digital 3D models of a patient's upper and lower jaw using one or more imaging devices.
b. Using artificial intelligence algorithms to align the digital 3D models of the upper and lower jaw, taking into account translation, rotation, and degrees of freedom.
c. Performing an occlusion and penetration analysis to determine the optimal bite position by adjusting the contact points between the teeth.
d. Generating a dynamic bite simulation to replicate the patient-specific jaw movements for bite analysis.

The following additional features may also be provided:
Repair of digital models, closing of holes, and removal of artifacts to improve model quality and/or
Reducing file sizes using a proprietary compression algorithm while maintaining model integrity.
Aspects, whereby dynamic bite simulation includes the replication of real activities such as chewing and speaking in order to evaluate the functionality of the bite. and/or
automating the preparation of digital models for 3D printing and/or
automated quality control checks are performed to ensure that bite adjustments and models meet predefined quality standards and/or
a computer program product comprising computer-readable instructions stored on a non-transitory computer-readable medium for performing the method described above and/or
an artificial intelligence-controlled system for creating a digital 3D model of a patient's bite, comprising
a. One or more imaging devices for capturing digital 3D models of a patient's upper and lower jaw.
b. A processing unit configured to align the digital 3D models of the upper and lower jaw using artificial intelligence algorithms, taking into account translation, rotation, and degrees of freedom.
c. An analysis module for performing occlusion and penetration analyses to determine the optimal bite alignment by adjusting the contact points between the teeth.

This can be supplemented in accordance with aspects of the invention by:
a dynamic simulation module for generating a dynamic bite simulation to replicate patient-specific jaw movements for bite analysis, and/or
a quality control module for automating quality control checks to ensure that bite adjustments and models meet predefined quality standards, and/or
a module for repairing digital models, closing holes, and removing artifacts to improve model quality, and/or
a module for reducing file size using a proprietary compression algorithm while maintaining model integrity, and/or
an aspect wherein the dynamic simulation module provides the ability to simulate real-life activities such as chewing and speaking to evaluate bite functionality and/or
a module for automating the preparation of digital models for 3D printing, and/or
a user interface for dental professionals to interact with the system.

Further Advantages of the Invention Can Be Seen in:
1. Improved accuracy: Bite-Finder uses advanced artificial intelligence (Al) algorithms to ensure highly accurate bite adjustments and reduce the risk of errors and remakes.
2. Efficiency gains: The system significantly reduces the time required for bite analysis and treatment planning, improving the efficiency of the entire workflow.
3. Cost savings: Fewer manual adjustments, less material waste, and improved workflow efficiency result in cost savings for dental practices and laboratories.
4. Improved patient comfort: Precise bite adjustments result in improved patient comfort during procedures and treatment.
5. Versatility: Bite-Finder can be used in various dental specialties, from general dentistry to orthodontics and prosthetics, making it a versatile tool for dentists.
6. Streamlined workflow: The system automates complex processes such as model repair, hole filling, artifact removal, and file size reduction, simplifying the workflow for dentists and dental technicians.
7. Automated model preparation: Bite-Finder automates the preparation of digital models for 3D printing, saving time and reducing the need for manual intervention.
8. Quality control: Automatic quality controls ensure that bite adjustments and models meet industrial and clinical standards, reducing the likelihood of suboptimal results.
9. Error reduction: By minimizing manual intervention and reliance on human judgment, Bite-Finder reduces the potential for human error, leading to more consistent results.
10. Cost-efficient CAD/CAM processing: The proprietary compression algorithm reduces file sizes without compromising model quality, enabling more cost-efficient CAD/CAM processing.
11. Responds to industry challenges: Bite-Finder addresses existing challenges in the dental industry, such as the need for more accurate bite modeling and the demand for automation to counteract labor shortages.
12. Improved treatment planning: The dynamic bite simulation feature allows dentists to evaluate bite functionality during real-life activities such as chewing and speaking, resulting in improved treatment planning.
13. Patient satisfaction: Precise bite adjustments improve the fit and comfort of braces, leading to higher patient satisfaction and fewer complaints after treatment.
14. Real-time feedback: Dentists can receive real-time feedback on bite adjustments and treatment plans, enabling quick adjustments and improvements.
15. Versatile applications: Bite-Finder can be used for various dental procedures, including restorative treatments, orthodontics, implantology, and more, making it a valuable tool in a range of dental practices.
16. Integration capabilities: The system can be integrated with other dental technologies such as 2D and 3D imaging, clinical photos, and facial scans to enrich information and improve results.
17. Potential for further innovation: The modular design of Bite-Finder allows for the integration of additional algorithms and features, which may pave the way for further innovation in the dental field.

Preferred fields of application and areas of use can be seen in:
1. Prosthetics:
Precise bite corrections for the fabrication of crowns, bridges, and prostheses.
Evaluation and correction of occlusal discrepancies in prosthetic treatments.
2. Orthodontics:
Assessment of bite relationships in orthodontic cases.
Simulation of jaw movements for planning orthodontic treatments.
3. Implantology:
Analysis of occlusal fit for implant-supported restorations.
Ensuring optimal occlusion for implant planning.
4. Restorative dentistry:
Bite analysis for restorative procedures such as fillings and veneers.
Checking the occlusal fit of restorative materials.
5. Occlusion analysis:
Comprehensive occlusion analysis to determine bite discrepancies.
Identification and correction of occlusal interferences.
6. Temporomandibular joint disorders (TMJ):
Evaluation of jaw movements and bite relationships in cases of temporomandibular joint disorders.
Planning treatments to fix jaw joint problems.
7. Treatment planning:
Simulation of bite functionality during treatment planning.
Optimization of treatment plans based on dynamic bite analysis.
8. Post-treatment assessment:
Checking bite accuracy and comfort after dental treatment.
Adjustments and refinements based on bite analysis after treatment.
9. Prosthetic restoration:
Bite-Finder can be used to optimize the fit and comfort of various prosthetic restorations, including full arch reconstructions and partial dentures.
10. Removable Appliances:
Analysis of bite relationships in removable appliances such as partial dentures.
Ensuring a comfortable fit for patients with removable dentures.
11. Quality control:
Ongoing quality control in dental laboratories to verify the accuracy of digital models.
Identification and correction of problems in digital models before they lead to clinical problems.
12. Dental education:
Training and education of dental students and professionals in bite analysis and adjustment.
Simulation and teaching of bite-related concepts in a virtual environment.
13. Integration with CAD/CAM:
Integration with computer-aided design and computer-aided manufacturing (CAD/CAM) for a seamless digital workflow in dental laboratories.
14. Collaboration with other professionals:
Facilitating collaboration between dentists, e.g., orthodontists, prosthodontists, and oral surgeons, by providing a common digital platform for bite analysis.
15. Research and development:
Support dental research and development by providing accurate bite modeling and analysis capabilities for studies and experiments.
16. Post-traumatic cases:
Assessment and correction of bite problems resulting from dental trauma.
Restoring bite function and aesthetics in post-traumatic cases.
17. Pediatric dentistry:
Analysis and adjustment of the bite in pediatric patients for various dental procedures.
Ensuring proper occlusion and alignment in growing children.
18. Multidisciplinary cases:
Involvement in complex multidisciplinary cases where several dentists are involved in treatment planning and implementation.
19. Continuous monitoring:
Continuous bite monitoring in patients with dentures to ensure long-term comfort and effectiveness.

1. Method for creating a digital 3D model of a patient's dentition, in which, in a computing unit, for a plurality of dentition variants, each of which comprising a combination of a digital 3D model of the upper jaw and a digital 3D model of the lower jaw of the patient, and which differ from one another in the relative positioning of the upper and lower jaws, the following is performed in each case:
the digital 3D model of the upper jaw is combined with the digital 3D model of the patient's lower jaw in such a way that the upper and lower jaws rest on each other at a number of contact points without any spatial overlap of parts of the upper and lower jaws, and
the contact surface between the upper and lower jaw is determined, wherein, taking into account the contact surface, an optimized dentition variant is selected as a 3D model of the dentition.

2. Method according to claim 1, in which dentition variants are taken into account that differ from one another in the inclination of the upper jaw relative to the lower jaw.

3. Method according to claim 1, wherein the dentition variant is selected for which the contact surface between the upper and lower jaw assumes a maximum value.

4. Method according to claim 1, in which a plurality of dentition variants corresponding to a temporal sequence within a chewing movement are taken into account for the selection of the 3D model of the dentition.

5. Method according to claim 1, wherein the creation of the dentition variants and/or the determination of the dentition variant to be selected is carried out by means of artificial intelligence.

6. Automatic analysis system, in particular for carrying out the method according to claim 1, comprising:
a first computing device comprising a memory that stores non-transitory instructions which, when executed by one or more processors of the first computing device, cause the first computing device: to read out the digital 3D models of the upper jaw and the lower jaw from a second computing device comprising a mass storage device in which a digital 3D model of the upper jaw of a patient and a digital 3D model of the lower jaw of the patient are stored, for a plurality of dental variants, each comprising a combination of the digital 3D model of the upper jaw with the digital 3D model of the lower jaw of the patient, which differ from one another in the relative positioning of the upper and lower jaws with respect to one another, in each case:
to combine the digital 3D model of the upper jaw with the digital 3D model of the patient's lower jaw in such a way that the upper and lower jaws rest on each other at a number of contact points without any spatial overlap of parts of the upper and lower jaws, and
to determine the contact surface between the upper and lower jaw, and, taking into account the contact surface, to select an optimized dentition variant as a 3D model of the dentition.

7. System according to claim 6, wherein the non-transitory commands, when executed by one or more processors of the first computing device, cause the first computing device to select the dentition variant as a 3D model of the dentition for which the contact surface of the upper and lower jaws assumes a maximum value.

8. System according to claim 6, wherein the non-transitory commands, when executed by one or more processors of the first computing device, cause the first computing device to take into account dentition variants that differ from each other in the inclination of the upper jaw relative to the lower jaw.

9. System according to claim 6, wherein the non-transitory commands, when executed by one or more processors of the first computing device, cause the first computing device to take into account a plurality of dentition variants corresponding to a temporal sequence within a chewing movement for the selection of the 3D model of the dentition.

10. (canceled)
